(19) United States
(12) Patent Application Publication
(10) Pub. No.: US 2009/0148917 A1
(43) Pub. Date: Jun. 11, 2009

(54) METHOD FOR PRODUCING CHIRAL ALCOHOLS

(75) Inventors: Antje Gupta, Wiesbaden (DE); Maria Bobkova, Idstein (DE); Anke Tschentscher, Eltville-Hattenheim (DE)

Correspondence Address: Workman Nydegger;1000 Eagle Gate Tower, 60 East South Temple, Salt Lake City, UT, 84111, US

(73) Assignee: IEP GMBH, Wiesbaden (DE)

(21) Appl. No.: 11/718,118

(22) PCT Filed: Oct. 26, 2005
(86) PCT No.: PCT/EP05/11459

§ 371 Date: Apr. 27, 2007

(30) Foreign Application Priority Data
Oct. 27, 2004 (AT) A 1808/2004

(51) Int. Cl. C12P 7/04; C12P 13/02

(52) U.S. Cl. 435/129; 435/157

(57) ABSTRACT

The invention relates to a method for producing an enantiopure alcohol of general formula (Ia) or (Ib), wherein R₁, R₂, R₃, R₄, R₅ and R₆ each represent hydrogen, halogen, a C₁-C₆ alkyl or C₁-C₆ alkoxy group, with the proviso that at least one of the groups R₁, R₂, R₃, R₄, R₅ and R₆ is different from the remaining five groups and with the additional proviso that at least one of the groups R₁, R₂, R₃, R₄, R₅ and R₆ is a halogen. The invention is characterized in that a ketone of general formula (II), wherein R₁, R₂, R₃, R₄, R₅ and R₆ are defined as above, is enzymatically reduced in the presence of an S-specific or R-specific dehydrogenase/oxidoreductase using NADH or NADPH as the cofactor.

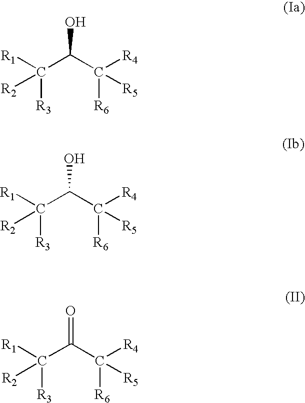

The invention relates to a method of producing enantiopure alcohols of general formula Ia or Ib, respectively,

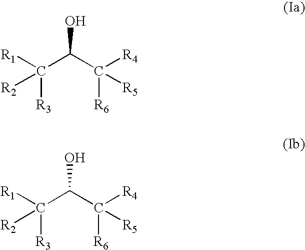

wherein R₁, R₂, R₃, R₄, R₅ and R₆ each represent hydrogen, halogen, a C₁-C₆ alkyl or C₁-C₆ alkoxy group, with the proviso that at least one of the moieties R₁, R₂, R₃, R₄, R₅ and R₆ is different from the remaining five moieties and with the additional proviso that at least one of the moieties R₁, R₂, R₃, R₄, R₅ and R₆ is a halogen.

Furthermore, the invention relates to a method of producing enantiopure alcohols of general formula IIIa or IIIb, respectively,

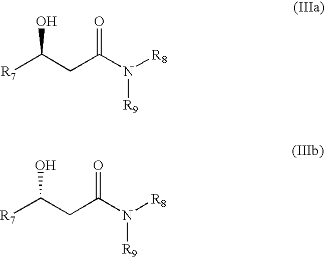

wherein R₇, R₈ and R₉ represent a C₁-C₆ alkyl group.

Enantiopure alcohols of the general formulae Ia or Ib, respectively, and IIIa or IIIb, respectively, constitute valuable chirons for the synthesis of a plurality of chiral compounds which are of interest for the production of pharmaceutically active substances. However, many of those enantiopure alcohols are not obtainable at all via a chemical route, or only in a very complex manner, and thus are not available in larger amounts.

It is therefore an object of the invention to provide a method which enables the economic production of enantiopure alcohols of the general formulae Ia or Ib, respectively, and IIIa or IIIb, respectively, in high yields and with high enantiopurity.

According to the invention, said object is achieved with respect to alcohols of general formula Ia or Ib, respectively, in that a ketone of general formula II

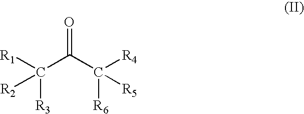

wherein R₁, R₂, R₃, R₄, R₅ and R₆ each represent hydrogen, halogen, a C₁-C₆ alkyl or C₁-C₆ alkoxy group, with the proviso that at least one of the moieties R₁, R₂, R₃, R₄, R₅ and R₆ is different from the remaining five moieties and with the additional proviso that at least one of the moieties R₁, R₂, R₃, R₄, R₅ and R₆ is a halogen, is enzymatically reduced in the presence of an S-specific or R-specific dehydrogenase/oxidoreductase using NADH or NADPH as the cofactor.

A preferred embodiment of the method is characterized in that R₁=R₂=Cl and R₃=R₄=R₅=R₆=H.

Another preferred embodiment is characterized in that R₁=R₂=R₄=Cl and R₃=R₅=R₆=H.

A further preferred embodiment is characterized in that R₁=CH₃, R₂=Cl and R₃=R₄=R₅=R₆=H.

Yet another preferred embodiment is characterized in that R₁=Cl and R₂=R₃=R₄=R₅=R₆=H.

The problem underlying the invention with respect to alcohols of general formula IIIa or IIIb, respectively, is solved in that a ketone of general formula IV

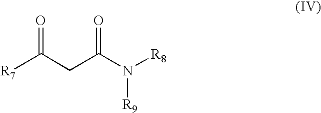

wherein R₇, R₈ and R₉ represent a C₁-C₆ alkyl group, is enzymatically reduced in the presence of an S-specific or R-specific dehydrogenase/oxidoreductase using NADH or NADPH as the cofactor.

By the term “NADH”, reduced nicotinamide adenine dinucleotide is understood, and by the term “NAD”, nicotinamide adenine dinucleotide is understood. By the term “NADPH”, reduced nicotinamide adenine dinucleotide phosphate is understood, and by the term “NADP”, nicotinamide adenine dinucleotide phosphate is understood.

A preferred embodiment of said method is characterized in that R₇=R₈=R₉=CH₃.

Another preferred embodiment is characterized in that R₇=CH₃ and R₈=R₉=C₂H₅.

The ketones of general formula II or IV, respectively, which, according to the invention, serve as the starting material, are generally readily available at low cost.

According to a preferred embodiment, the dehydrogenase used for the enzymatic reduction is obtained from a microbial starting material. Which configuration of the products is predominantly or exclusively formed depends on the type of the dehydrogenase/oxidoreductase and also on the type of the cofactor.

In the methods of producing enantiopure alcohols of the general formulae Ia or Ib, respectively, and IIIa or IIIb, respectively, a secondary alcohol dehydrogenase from lactobacteria of the genus Lactobacilliales, in particular Lactobacillus kefir, Lactobacillus brevis or Lactobacillus minor, or from Pseudomonas is preferably used as the R-specific dehydrogenase.

Thereby, under R-specific secondary alcohol dehydrogenases are understood those which reduce the keto group in a grouping H₃C—C(C═O)—CH₂—C to the corresponding (R)-configured alcohol. Such R-specific secondary alcohol dehydrogenases are described, for instance, in U.S. Pat. No. 5,200,335, DE 196 10 984 A1, DE 101 19 274 or U.S. Pat. No. 5,385,833.

A secondary alcohol dehydrogenase of the genus Pichia or Candida, in particular Candida boidinii ADH, Candida parapsilosis or Pichia capsulata, is preferably used as the S-specific dehydrogenase. Such S-specific dehydrogenases are described, for instance, in U.S. Pat. No. 5,523,223 or DE 103 27 454.

The enzyme does not have to be used in the pure form. Enzyme-containing microorganisms. or lysates thereof which have been purified more or less can be used just as well. If the reaction is to be carried out continuously, immobilized enzymes can also be used. Immobilization can be effeced, for example, by incorporating the enzymes particularly in polymeric networks or in semipermeable membranes or by binding them to a carrier, e.g., by absorption or by ionic or covalent bonds. However, the dehydrogenases are preferably used in the free form.

The enzymatic reduction itself proceeds under mild conditions so that the alcohols produced will not react further. The methods according to the invention exhibit a high dwelling time, an enantiopurity of more than 95% of the produced chiral alcohols of the formulae Ia or Ib, respectively, and IIIa or IIIb, respectively, and a high yield, based on the employed amount of keto compounds of formula II or IV, respectively.

In the methods according to the invention, the oxidoreductases can be used either in a completely purified or partially purified state, in the form of cell lysates or in the form of whole cells. The cells used can thereby be provided in the native or in a permeabilized state. Cloned and overexpressed oxidoreductases (known, e.g., from U.S. Pat. No. 5,523,223, DE 103 27 454 or DE 101 19 274) are preferably used.

According to a preferred embodiment of the methods, the volume activity of the oxidoreductase used ranges from 10 U/ml to 5000 U/ml, preferably from 100 U/ml to 1000 U/ml.

Per kg of ketone to be reduced, 5000 to 10.000.000 U, preferably 10.000 to 1.000.000 U, of oxidoreductase is used in said method. Thereby, the enzyme unit 1 U corresponds to the enzyme amount which is required for converting 1 μmol of the keto compound of formula II or IV, respectively, per minute.

Furthermore, a preferred embodiment of the invention is characterized in that the NAD or NADP formed during the reduction is continuously reduced with a cosubstrate to NADH or NADPH, respectively.

In doing so, primary and secondary alcohols such as ethanol, 2-propanol, 2-butanol, 2-pentanol, 4-methyl-2-pentanol, 2-octanol or cyclohexanol are preferably used as the cosubstrate.

Said cosubstrates are reacted to the corresponding aldehydes or ketones and NADH or NADPH, respectively, with the aid of an oxidoreductase and NAD or NADP, respectively. This results in a regeneration of the NADH or NADPH, respectively. The proportion of the cosubstrate for the regeneration hereby ranges from 5 to 95% by volume, based on the total volume.

For the regeneration of the cofactor, an additional alcohol dehydrogenase can be added. Suitable NADH-dependent alcohol dehydrogenases are obtainable, for example, from baker's yeast, from Candida boidinii, Candida parapsilosis or Pichia capsulata. Furthermore, suitable NADPH-dependent alcohol dehydrogenases are present in Lactobacillus brevis (DE 196 10 984 A1), Lactobacillus minor (DE 101 19 274), Pseudomonas (U.S. Pat. No. 5,385,833) or in Thermoanaerobium brockii. Suitable cosubstrates for these alcohol dehydrogenases are the already mentioned secondary alcohols such as ethanol, 2-propanol (isopropanol), 2-butanol, 2-pentanol, 4-methyl-2-pentanol, 2-octanol or cyclohexanol.

Furthermore, cofactor regeneration can also be effected, for example, using NAD- or NADP-dependent formate dehydrogenase (Tishkov et al., J. Biotechnol. Bioeng. [1999] 64, 187-193, Pilot-scale production and isolation of recombinant NAD and NADP specific Formate dehydrogenase). Suitable cosubstrates of formate dehydrogenase are, for example, salts of formic acid such as ammonium formate, sodium formate or calcium formate. However, the methods according to the invention are preferably carried out without such an additional dehydrogenase, i.e., substrate-coupled coenzyme regeneration takes place.

The aqueous portion of the reaction mixture in which the enzymatic reduction proceeds preferably contains a buffer, e.g., a potassium phosphate, tris/HCl or triethanolamine buffer, having a pH value of from 5 to 10, preferably a pH value of from 6 to 9. In addition, the buffer can comprise ions for stabilizing or activating the enzymes, for example, zinc ions or magnesium ions.

While carrying out the methods according to the invention, the temperature suitably ranges from about 10° C. to 70° C., preferably from 20° C. to 40° C.

In a further preferred embodiment of the methods according to the invention, the enzymatic conversion is effected in the presence of an organic solvent which is not or only partially miscible with water. Said solvent is, for example, a symmetric or unsymmetric di(C₁-C₆)alkyl ether, a straight-chain or branched alkane or cycloalkane or a water-insoluble secondary alcohol simultaneously representing the cosubstrate. The preferred organic solvents are, for example, diethyl ether, tertiary butyl methyl ether, diisopropyl ether, dibutyl ether, butyl acetate, heptane, hexane, 2-octanol, 2-heptanol, 4-methyl-2-pentanol or cyclohexane.

If water-insoluble solvents and cosubstrates, respectively, are used, the reaction batch consists of an aqueous and an organic phase. The substrate is distributed between the organic and the aqueous phase according to its solubility. The organic phase generally has a proportion of from 5 to 95%, preferably from 20 to 90%, based on the total reaction volume. The two liquid phases are preferably mixed mechanically so that a large surface is produced between them. Also in this embodiment, the NAD or NADP, respectively, formed during the enzymatic reduction can be reduced back to NADH or NADPH, respectively, using a cosubstrate, as described above.

The concentration of the cofactor NADH or NADPH, respectively, in the aqueous phase generally ranges from 0.001 mM to 1 mM, in particular from 0.01 mM to 0.1 mM.

In the methods according to the invention, a stabilizer of the oxidoreductase/dehydrogenase can, in addition, be used. Suitable stabilizers are, for example, glycerol, sorbitol, 1,4-DL-dithiothreitol (DTT) or dimethyl sulfoxide (DMSO).

The method according to the invention is carried out, for example, in a closed reaction vessel made of glass or metal. For this purpose, the components are transferred individually into the reaction vessel and stirred under an atmosphere of, e.g., nitrogen or air. The reaction time ranges from 1 hour to 48 hours, in particular from 2 hours to 24 hours.

Subsequently, the reaction mixture is processed. For this purpose, the aqueous phase is separated, the organic phase is filtered. The aqueous phase can optionally be extracted once more and can be processed further like the organic phase. Thereupon, the solvent is optionally evaporated from the filtered organic phase.

Below, the invention is illustrated in further detail by way of examples.

### EXAMPLES

### Analytics:

### a) Amides:

The determination of the ee (enantiomeric excess) was performed via chiral gas chromatography. For this purpose, a gas chromatograph GC-17A of Shimadzu was used with a chiral separating column CP-Chirasil-DEX CB (Varian Chrompack, Darmstadt, Germany), a flame ionization detector and helium as a carrier gas.
The separation of N,N-dimethyl-3-hydroxybutanamide was effected at 0.86 bar and for 10 min at 120° C., 2° C./min→125° C.
The retention times were: (3R) 10.42 min and (3S) 10.09 min.
The separation of N,N-diethyl-3-hydroxybutanamide was effected at 0.75 bar and for 10 min at 130° C., 2° C./min→135° C.
The retention times were: (3R) 11.6 min and (3S) 11.3 min.

### b) Chlorine Compounds:

The determination of the ee (enantiomeric excess) was performed via chiral gas chromatography. For this purpose, a gas chromatograph GC-17A of Shimadzu was used with a chiral separating column FS-Hydrodex β-6-TBDM (Machery-Nagel, Düren, Germany), a flame ionization detector and helium as a carrier gas.
The separation of 1-chloropropane-2-ol was effected at 0.94 bar and for 15 min at 40° C., 1° C./min→50° C.
The retention times were: (2R) 20.3 min and (2S) 20.9 min.
The separation of 1,1-dichloropropane-2-ol was effected at 0.69 bar and for 15 min at 80° C., 2° C./min→95° C.
The retention times were: (2R) 20.8 min and (2S) 21.4 min.
The separation of 1,1,3-trichloropropane-2-ol was effected at 0.69 bar and for 30 min at 120° C. isothermally.
The retention times were: (2R) 25.0 min and (2S) 24.5 min.
The separation of 3-chlorobutane-2-ol was effected at 0.98 bar and for 25 min at 50° C. isothermally.
The retention times were:

### (3R)-3-Chlorobutane-2-one: 6.0 min

### (3S)-3-Chlorobutane-2-one: 6.2 min

### (3R,2R)-3-Chlorobutane-2-ol: 17.5 min

### (3R,2S)-3-Chlorobutane-2-ol: 18.1 min

### (3S,2R)-3-Chlorobutane-2-ol: 20.7 min

### (3S,2S)-3-Chlorobutane-2-ol: 22.1 min

1. Synthesis of (S)-3-hydroxy-N,N-diethylbutanamide from N,N-diethyl-acetoacetamide

For the synthesis of (R)-3-hydroxy-N,N-diethylbutanamide, a mixture of 172 ml buffer (100 mM triethanolamine, pH=7, 0.5 mM DTT, 20% glycerol), 18 ml 2-propanol (0.23 mol), 10 ml N,N-diethylacetoacetamide (63 mmol), 200 mg NAD and 30000 units of recombinant alcohol dehydrogenase from Candida parapsilosis was incubated at room temperature for 24 h under constant mixing. After 24 h, 97% of the N,N-diethylacetoacetamide used had been reduced to (S)-3-hydroxy-N,N-diethylbutanamide. Subsequently, the reaction mixture was extracted with ethyl acetate and the solvent was removed on the rotary evaporator. The crude product thus obtained was purified by vacuum distillation. 2.5 g (S)-3-hydroxy-N,N-diethylbutanamide having a purity of >98% and an enantiomeric excess of >99.9% could be obtained.

### Analytical Results:

Elemental analysis % found (calculated): C₈H₁₇NO₂

### C: 59.8 (60.4)

### H: 10.7 (10.8)

### N: 8.9 (8.8)

### ¹H-NMR in CDCl₃:

| Signal | Integral | Allocation |
|---|---|---|
| 1.13 (T) + 1.19 ppm (T) | 6 | 2x CH₃ (ethyl) |
| 1.22 ppm | 3 | CH₃ (adjacent the chiral centre) |
| 2.3 (DvD) + 2.5 (DvD) | 2 | CH₂ (adjacent the chiral centre) |
| 3.2 (DvD) + 3.5 (DvD) | 4.2 | 2 x CH₂ |
| 4.2 (M) | 1 | CH (chiral centre) |
| 4.7 (S) | 0.9 | OH |

### ¹³C-NMR in CDCl₃:

| Signal | Allocation |
|---|---|
| 12 ppm | CH₃ (ethyl) |
| 14 ppm | CH₃ (ethyl) |
| 22 ppm | CH₃ (adjacent the chiral centre) |
| 39 ppm | CH₂ |
| 40 ppm | CH₂ |
| 41 ppm | CH₂ |
| 64 ppm | CH (chiral centre) |
| 171 ppm | C═O |
| (78 ppm) | solvent (CDCl₃) |

### Specific Amounts of Rotation:

The specific amount of rotation [α]_{D}²⁰ of the enantiomers was measured with a precision polarimeter POL-S2 at a layer thickness of 1 dm. For the assay, 0.5 g of the sample was dissolved in 25 ml EtOH.
(S)-3-Hydroxy-N,N-diethylbutanamide (100%) [α]_{D}²⁰ =+19.92±1°×1/g·dm
2. Synthesis of (R)-3-hydroxy-N,N-diethylbutanamide from N,N-diethylacetoacetamide

For the synthesis of (R)-3-hydroxy-N,N-diethylbutanamide, a mixture of 290 ml buffer (100 mM triethanolamine, pH=7, 1 mM MgCl₂, 10% glycerol), 100 ml 2-propanol (1.3 mol), 10 ml N,N-diethylacetoacetamide (63 mmol), 20 mg NADP and 60000 units of recombinant alcohol dehydrogenase from Lactobacillus minor (DE-A 101 19 274) was incubated at room temperature for 24 h under constant mixing. After 24 h, 60% of the N,N-diethyl-acetoacetamide used had been reduced to (R)-3-hydroxy-N,N-diethylbutanamide. Subsequently, the reaction mixture was extracted with ethyl acetate and the solvent was removed on the rotary evaporator. The crude product thus obtained was purified by vacuum distillation. 2.5 g (R)-3-hydroxy-N,N-diethylbutanamide having a purity of >98% and an enantiomeric excess of >99% could be obtained.

### Analytical Results:

Elemental analysis % found (calculated): C₈H₁₇NO₂

### C: 59.8 (60.4)

### H: 10.7 (10.8)

### N: 8.9 (8.8)

¹H-NMR in CDCl₃: results analogous to Example 1
¹³C-NMR in CDCl₃: results analogous to Example 1

### Specific Amounts of Rotation:

(R)-3-Hydroxy-N,N-diethylbutanamide (100%) [α]_{D}²⁰=−19.7±1°×1/g·dm

Via ¹H-NMR, ¹³ C-NMR and elemental analysis, the structure of the alcohols (S)- and (R)-3-hydroxy-N,N-diethylbutanamide could be verified. The precise determination of the enantiomeric excess was performed via chiral GC and by determining the amount of rotation [α]_{D}²⁰.
3. Synthesis of (R)-3-hydroxy-N,N-dimethylbutanamide from N,N-dimethylacetoacetamide

For the synthesis of (R)-3-hydroxy-N,N-dimethylbutanamide, a mixture of 525 ml buffer (100 mM triethanolamine, pH=7, 1 mM MgCl₂, 10% glycerol), 90 ml 2-propanol (1.18 mol), 15 ml N,N-dimethylacetoacetamide (120 mmol), 30 mg NADP and 50000 units of recombinant alcohol dehydrogenase from Lactobacillus minor (DE-A 101 19 274) was incubated at room temperature for 24 h under constant mixing. After 24 h, 95% of the N,N-dimethylacetoacetamide used had been reduced to (R)-3-hydroxy-N,N-diethylbutanamide. Subsequently, the reaction mixture was extracted with ethyl acetate and the solvent was removed on the rotary evaporator. The crude product thus obtained was purified by vacuum distillation. 2.3 g (R)-3-hydroxy-N,N-dimethylbutanamide having a purity of >98% and an enantiomeric excess of >99.0% could be obtained.

### Analytical Results:

Elemental analysis % found (calculated): C₆H₁₃NO₂

### C: 54.2 (54.9)

### H: 9.7 (10.0)

### N: 10.4 (10.7)

### ¹H-NMR in CDCl₃:

| Signal | Integral | Allocation |
|---|---|---|
| 1.2 ppm (D) | 3 | CH₃ (adjacent the chiral centre) |
| 2.3 (DvD) + 2.5 (DvD) ppm | 2 | CH₂ (adjacent the chiral centre) |
| 2.9 (S) + 3.0 (S) ppm | 6 | 2 x CH₃ |
| 4.2 ppm (M) | 1 | CH (chiral centre) |
| 4.6 ppm (S) | 1.0 | OH |

### ¹³C-NMR in CDCl₃:

| Signal | Allocation |
|---|---|
| 22 ppm | CH₃ (adjacent the chiral centre) |
| 34 ppm | CH₃ |
| 36 ppm | CH₃ |
| 41 ppm | CH₂ |
| 64 ppm | CH (chiral centre) |
| 171 ppm | C═O |
| (78 ppm) | solvent (CDCl₃) |

### Specific Amounts of Rotation:

(R)-3-Hydroxy-N,N-dimethylbutanamide (100%) [α]_{D}²⁰=−29.1±1°×1/g·dm
4. Synthesis of (S)-3-hydroxy-N,N-dimethylbutanamide from N,N-dimethylacetoacetamide

For the synthesis of (S)-3-hydroxy-N,N-dimethylbutanamide, a mixture of 89 ml buffer (100 mM triethanolamine, pH=7, 1 mM ZnCl₂, 10% glycerol), 9 ml 2-propanol (0.12 mol), 2.5 ml N,N-dimethylacetoacetamide (19 mmol), 10 mg NAD and 16000 units of recombinant alcohol dehydrogenase from Candida parapsilosis was incubated at room temperature for 24 h under constant mixing. After 24 h, 95% of the N,N-dimethylacetoacetamide used had been reduced to (S)-3-hydroxy-N,N-diethylbutanamide. Subsequently, the reaction mixture was extracted with ethyl acetate and the solvent was removed on the rotary evaporator. The crude product thus obtained was purified by vacuum distillation. (S)-3-Hydroxy-N,N-dimethylbutanamide having a purity of >98% and an enantiomeric excess of >99.0% could thus be obtained.

### Specific Amounts of Rotation:

(S)-3-Hydroxy-N,N-dimethylbutanamide (100%) [α]_{D}²⁰=+29.1±1°×1/g·dm
5. Synthesis of (S)-1,1-dichloro-2-propanol from 1,1-dichloroacetone

For the synthesis of (S)-1,1-dichloro-2-propanol, a mixture of 640 ml buffer (100 mM triethanolamine, pH=7, 1 mM ZnCl₂, 20% glycerol), 80 ml 2-propanol (1.05 mol), 20 ml 1,1-dichloroacetone (0.2 mol), 40 mg NAD and 13000 units of recombinant alcohol dehydrogenase from Pichia capsulata (DE-A 103 27 454) was incubated at room temperature for 24 h under constant mixing. After 24 h, 100% of the 1,1-dichloroacetone used had been reduced to (S)-1,1-dichloro-2-propanol. Subsequently, the reaction mixture was extracted with ethyl acetate and the solvent was removed on the rotary evaporator. The crude product thus obtained was purified by vacuum distillation. 6.5 g (S)-1,1-dichloro-2-propanol having a purity of >99% and an enantiomeric excess of >99.9% could be obtained.

Analytical Results:

Elemental analysis and chlorine determination % found (calculated): C₃H₆Cl₂O

### C: 26.7 (27.9)

### H: 4.7 (4.7)

### O: 14.8 (12.4)

### Cl: 53.4 (55)

### ¹H-NMR in CDCl₃:

| Signal | Integral | Allocation |
|---|---|---|
| 1.3 ppm (D) | 3.1 | CH₃ |
| 3.2 ppm (D) | 1 | OH |
| 4.1 ppm (DvQ) | 1 | CH (chiral centre) |
| 5.7 ppm (S) | 1.0 | CH |

### ¹³C-NMR in CDCl₃:

| Signal | Allocation |
|---|---|
| 18 ppm | CH₃ |
| 72 ppm | CH |
| 77 ppm | CH |

### Specific Amounts of Rotation:

(S)-1,1-Dichloro-2-propanol (100%) [α]_{D}²⁰=−19.1±1°×1/g·dm
6. Synthesis of (R)-1,1-dichloro-2-propanol from 1,1-dichloroacetone

For the synthesis of (R)-1,1-dichloro-2-propanol, a mixture of 320 ml buffer (100 mM triethanolamine, pH=7, 1 mM MgCl₂, 10% glycerol), 60 ml 2-propanol (0.78 mol), 20 ml 1,1 -dichloroacetone (0.2 mol) dissolved in 40 ml ethyl acetate, 40 mg NADP and 8000 units of recombinant alcohol dehydrogenase from Lactobacillus minor (DE-A 101 19 274) was incubated at room temperature for 24 h under constant mixing. After 24 h, 100% of the 1,1 -dichloroacetone used had been reduced to (R)-1,1-dichloro-2-propanol. Subsequently, the reaction mixture was extracted with ethyl acetate and the solvent was removed on the rotary evaporator. The crude product thus obtained was purified by vacuum distillation. 4.8 g (R)-1,1-dichloro-2-propanol having a purity of >98% and an enantiomeric excess of >95% could be obtained.

### Analytical Results:

Elemental analysis and chlorine determination % found (calculated): C₃H₆Cl₂O

### C: 26.7 (27.9)

### H: 4.7 (4.7)

### O: 14.8 (12.4)

### Cl: 53.4 (55)

¹H-NMR in CDCl₃: analogous to Example 5
¹³C-NMR in CDCl₃: analogous to Example 5

### Specific Amounts of Rotation:

(R)-1,1-Dichloro-2-propanol (100%) [α]_{D}²⁰=+19.56±1°×1/g·dm
7. Synthesis of (R)-1,1,3-trichloro-2-propanol from 1,1,3-trichloroacetone

For the synthesis of (R)-1,1,3-trichloro-2-propanol, a mixture of 110 ml buffer (100 mM triethanolamine, pH=7, 1 mM MgCl₂, 10% glycerol), 40 ml 2-propanol (0.52 mol), 10 ml 1,1,3-trichloroacetone (93 mmol) dissolved in 40 ml ethyl acetate, 20 mg NADP and 12000 units of recombinant alcohol dehydrogenase from Lactobacillus minor (DE-A 101 19 274) was incubated at room temperature for 24 h under constant mixing. After 24 h, 100% of the 1,1,3-trichloroacetone used had been reduced to (R)-1,1,3-trichloro-2-propanol. Subsequently, the reaction mixture was extracted with ethyl acetate and the solvent was removed on the rotary evaporator. The crude product thus obtained was purified by vacuum distillation. 8.9 g (R)-1,1,3-trichloro-2-propanol having a purity of >99% and an enantiomeric excess of >97% could be obtained.

### Analytical Results:

Elemental analysis and chlorine determination % found (calculated): C₃H₅Cl₃O

### C: 22.1 (22.1)

### H: 2.8 (3.1)

### O: 11.1 (9.8)

### Cl: 63.9 (65.1)

### ¹H-NMR in CDCl₃:

| Signal | Integral | Allocation |
|---|---|---|
| 3.3 ppm (S) | 1 | OH |
| 3.8 ppm (D) | 2 | CH₂ |
| 4.2 ppm (M) | 1 | CH (chiral centre) |
| 5.9 ppm (D) | 1.0 | CH |

### ¹³C-NMR in CDCl₃:

| Signal | Allocation |
|---|---|
| 45 ppm | CH₂ |
| 73 ppm | CH |
| 77 ppm | CH |

### Specific Amounts of Rotation:

(R)-1,1,3-Trichloro-2-propanol (100%) [α]_{D}²⁰=+10.1±1°×1/g·dm
8. Synthesis of (S)-1,1,3-trichloro-2-propanol from 1,1,3-trichloroacetone

For the synthesis of (S)-1,1,3-trichloro-2-propanol, a mixture of 9 ml buffer (100 mM triethanolamine, pH=7, 1 mM ZnCl₂, 20% glycerol), 0.8 ml 2-propanol (10 mmol), 0.25 ml 1,1,3-trichloroacetone (0.2 mol), 10 mg NAD and 2000 units of recombinant alcohol dehydrogenase from Pichia capsulata (DE-A 103 27 454) was incubated at room temperature for 24 h under constant mixing. After 24 h, 97% of the 1,1,3-trichloroacetone used had been reduced to (S)-1,1,3-trichloro-2-propanol with an enantiomeric excess of >60%.

### Specific Amounts of Rotation:

(S)-1,1,3-Trichloro-2-propanol (100%) [α]_{D}²⁰=−10.1±1°×1/g·dm
9. Synthesis of S-chloro-2-propanol from chloroacetone

For the synthesis of S-chloro-2-propanol, a mixture of 0.6 ml buffer (100 mM triethanolamine, pH=7, 10% glycerol, 1 mM ZnCl₂), 400 μl 4-methyl-2-propanol, 100 μl chloroacetone, 1 mg NAD and 60 units of recombinant alcohol dehydrogenase from Pichia capsulata (DE-A 103 27 454) or Candida parapsilosis, respectively, was incubated at room temperature for 24 h under constant mixing. After 24 h, 100% of the chloroacetone used had been reduced to S-chloro-2-propanol with an enantiomeric excess of >97%.
10. Synthesis of R-chloro-2-propanol from chloroacetone

For the synthesis of R-chloro-2-propanol, a mixture of 0.4 ml buffer (100 mM triethanolamine, pH=7, 10% glycerol), 300 μl 2-propanol, 100 μl chloroacetone dissolved in 200 μl ethyl acetate, 1 mg NADP and 30 units of recombinant alcohol dehydrogenase from Lactobacillus minor (DE-A 101 19 274) was incubated at room temperature for 24 h under constant mixing. After 24 h, 100% of the chloroacetone used had been reduced to R-chloro-2-propanol with an enantiomeric excess of >95%.
11. Synthesis of (2S)-3-chloro-2-butanol from 3-chloro-2-butanone

For the synthesis of (2S)-3-chloro-2-butanol, a mixture of 0.45 ml buffer (100 mM triethanolamine, pH=7, 10% glycerol, 1 mM ZnCl₂), 450 μl 4-methyl-2-propanol, 100 μl 3-chloro-2-butanone (1 mmol), 0.1 mg NAD (0.15 μmol) and 60 units of recombinant alcohol dehydrogenase from Pichia capsulata (DE-A 103 27 454) or Candida parapsilosis, respectively, was incubated at room temperature for 24 h under constant mixing. After 24 h, 100% of the 3-chloro-2-butanone used had been reduced to (2S)-3-chloro-2-butanol with an enantiomeric excess of >98%.
12. Synthesis of (2R)-3-chloro-2-butanol from 3-chloro-2-butanone

For the synthesis of (2R)-3-chloro-2-butanol, a mixture of 0.45 ml buffer (100 mM triethanolamine, pH=7, 10% glycerol, 1 mM MgCl₂), 450 μl 4-methyl-2-propanol, 100 μl 3-chloro-2-butanone (1 mmol), 0.1 mg NADP (0.13 μmol) and 60 units of recombinant alcohol dehydrogenase from Lactobacillus minor (DE-A 101 19 274) was incubated at room temperature for 24 h under constant mixing. After 24 h, 100% of the 3-chloro-2-butanone used had been reduced to (2R)-3-chloro-2-butanol with an enantiomeric excess of >98%.

1. A method of producing an enantiopure alcohol of general formula Ia or Ib, respectively, 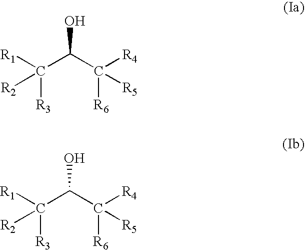
wherein R₁, R₂, R₃, R₄, R₅ and R₆ each represent hydrogen, halogen, a C₁-C₆ alkyl or C₁-C₆ alkoxy group, with the proviso that at least one of the moieties R₁, R₂, R₃, R₄, R₅ and R₆ is different from the remaining five moieties and with the additional proviso that at least one of the moieties R₁, R₂, R₃, R₄, R₅ and R₆ is a halogen, characterized in that a ketone of general formula II 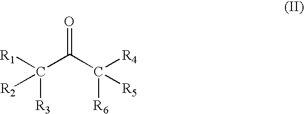
wherein R₁, R₂, R₃, R₄, R₅ and R₆ have the above indicated meaning, is enzymatically reduced in the presence of an S-specific or R-specific dehydrogenase/oxidoreductase using NADH or NADPH as the cofactor and that NAD or NADP formed during the reduction is continuously reduced with a secondary alcohol to NADH or NADPH, respectively.

2. The method according to claim 1, wherein R₁=R₂=Cl and R₃=R₄=R₅=R₆=H.

3. The method according to claim 1, wherein R₁=R₂=R₄=Cl and R₃=R₅=R₆=H.

4. The method according to claim 1, wherein R₁=CH₃, R₂=Cl and R₃=R₄=R₅=R₆=H.

5. The method according to claim 1, wherein R₁=Cl and R₂=R₃=R₄=R₅=R₆=H.

6.-8. (canceled)

9. The method according to claim 1, wherein a secondary alcohol dehydrogenase from lactobacteria of the genus Lactobacilliales, in particular Lactobacillus kefir, Lactobacillus brevis or Lactobacillus minor, or from Pseudomonas is used as the R-specific dehydrogenase.

10. The method according to claim 1, wherein a secondary alcohol dehydrogenase from the genus Pichia or Candida, in particular Candida boidinii ADH, Candida parapsilosis or Pichia capsulata, is used as the S-specific dehydrogenase.

11. The method according to claim 1, wherein the volume activity of the oxidoreductase used ranges from 10 U/ml to 5000 U/ml.

12. The method according to claim 1, wherein, per kg of ketone to be reduced, 5000 to 10.000.000 U, of oxidoreductase is used.

13. (canceled)

14. The method according to claim 1, wherein an alcohol from the group consisting of 2-propanol, 2-butanol, 2-pentanol, 4-methyl-2-pentanol, 2-octanol and cyclohexanol is used as the secondary alcohol.

15. The method according to claim 1, wherein the volume activity of the oxidoreductase used ranges from 100 U/ml to 1000 U/ml.

16. The method according to claim 1, wherein, per kg of ketone to be reduced, 10.000 to 1.000.000 U of oxidoreductase is used.
